(19) United States
(12) Patent Application Publication
(10) Pub. No.: US 2004/0076995 A1
(43) Pub. Date: Apr. 22, 2004

(54) Nurr1 transcription factor 1-box mutants having monomeric transcriptional activation activity

(76) Inventors: Thomas Perlmann, Stockholm (SE); Pila Aarnisalo, Helsinki (FI)

Correspondence Address: Mary Anne Schofield, Fulbright & Jaworski, Market Square, 801 Pennsylvania Avenue NW, Washington, DC, 20004-2615, US

(21) Appl. No.: 10/470,156

(22) PCT Filed: Jan. 25, 2002
(86) PCT No.: PCT/EP02/00744

(30) Foreign Application Priority Data
Jan. 26, 2001 (GB) 0102087.4

(51) Int. Cl. C12Q 1/68; C07H 21/04; C07K 14/705

(52) U.S. Cl. 435/6; 435/69.1; 435/320.1; 435/325; 530/350; 536/23.2

(57) ABSTRACT

The invention relates to the finding that the Nurr1 transciption factor, which forms a heterodimer with the retinoid X receptor, can be mutated in the I box region such that dimerisation does not occur while Nurr1 transcriptional activation activity is retained. The invention provides Nurr1 peptides with such I box mutations, as well as assay methods for modulators which affect the monomeric activity of Nurr1.

### FIELD OF THE INVENTION

The present invention relates to novel mutants of the Nurr1 transcription factor, and to their use in assays to identify modulators of Nurr1 activity.

### BACKGROUND OF THE INVENTION

Nurr1 is a transcription factor belonging to the superfamily of nuclear receptors. This receptor family consists of the receptors for steroid hormones, retinoids, thyroid hormone, vitamin D, and a large group of receptors whose ligands are unknown. The receptors with unidentified ligands are referred to as orphan nuclear receptors (Truss and Beato 1993, Mangelsdorf et al. 1995).

Most nuclear receptors act as dimers, either as homodimers or as heterodimers. Two dimerization interfaces have been identified in the DNA-binding domain and the ligand-binding domain (LBD), respectively. The dimerization interface in the LBD—the I-box—has been mapped to a region in the carboxyl-terminal part of the LBD, corresponding to helix 10 in the canonical nuclear receptor LBD structure (Perlmann et al. 1996, Lee et al. 1998). This region is well conserved among several dimerizing receptors, including Nurr1 (FIG. 1) and Nor 1. In Nurr1, the I-box is the region at amino acids 524-556 of the full length protein.

Nurr1 is able to bind to DNA as a monomer (Wilson et al. 1991). In addition, Nurr1 forms heterodimers with retinoid X receptor (RXR) and, in contrast to most other RXR-heterodimers, this dimer can be activated by retinoids (Perlmann and Jansson 1995, Forman et al 1995, Zetterström et al. 1996.

The functional roles of the Nurr1 monomer and Nurr1-RXR heterodimer in vivo remain to be elucidated. Nevertheless, expression studies relating to Nurr1 have shown that Nurr1, as the closely related receptors NGFI-B and Nor1, is predominantly expressed in the CNS (Milbrandt 1988, Law et al. 1992, Ohkura et al. 1994, Zetterström et al. 1996). In the CNS, it is detected e.g. in the cortex, hippocampus, hypothalamus, and in the dopaminergic neurons of the substantia nigra and ventral tegmental area (Zetterström et al. 1996).

Nurr1 is expressed in the developing and adult midbrain dopaminergic neurons. Studies have shown that Nurr1 plays a critical role in the development of these neurons as mice whose Nurr1 gene has been inactivated (Nurr1 −/− mice) fail to generate the midbrain dopamine cells (Zetterström et al. 1997, Saucedo-Cardenas et al. 1998, Castillo et al. 1998, Wallén et al. 1999). These neurons degenerate in patients with Parkinson's disease, and so Nurr1 may be important in the development of this disease. Nurr1 seems also to play an important role in other disorders of dopamine transmission as mutations in the coding sequence of Nurr1 have been identified in schizophrenic and manic-depressive patients (Buervenich et al. 2000).

Therefore, due to its potential role in dopaminergic neurons, Nurr1 is a potential target for pharmacological treatment of disorders involving dopamine transmission. Agents, for example small lipophilic compounds, which interact with Nurr1 and affect its transcriptional activation function or DNA binding properties have potential therapeutic utility.

Screens for compounds which modulate nuclear receptors are often based upon reporter gene activation assays performed in cultured cells. In such assays, it would be difficult to distinguish between agents which affect the activity of the Nurr1-RXR heterodimer, and those which are specific to the Nurr1 monomer. Thus, using wild-type Nurr1 in such an assay would require further testing of agents to determine which complex is modulated. Because it is possible that Nurr1 and Nurr1-RXR complexes have distinct roles in vivo, it would be desirable to be able to distinguish more efficiently between the monomer and the heterodimer complex.

The ligand binding domain of Nurr1, encompassing the I-box, is structurally complex. It has been shown that truncating a region including the I-box of Nor1 interferes with the monomeric activity of this receptor (Labelle et al, Oncogene 18(21):3303-8, 1999). By analogy, it might be expected that similar disruptions to Nurr1 would likewise disrupt the function of this protein.

### DISCLOSURE OF THE INVENTION

The present inventors have surprisingly discovered that Nurr1 polypeptides with particular mutations in the I-box region are unable to dimerise with RXR but surprisingly retain the ability to promote gene expression as monomers. This is particularly notable since the ligand binding domain of Nurr1 includes transcriptional activation functions, so that the removal of the ability to dimerise could lead to removal of transcription activation function.

The present invention thus provides a Nurr1 polypeptide which in comparison to a wild-type Nurr1 polypeptide has at least one amino acid difference, which is unable to promote RXR-mediated transactivation but which retains the ability to promote gene expression as a monomer, said difference being in the I-box region.

Preferred differences occur at one or more (e.g. two or three or four) I-box residues at residues 524 to 566, for example in the region of 553 to 563. Preferred residues are those selected from the positions 554, 555, 556, 557, 558, 559, 560, 561 and 562 of Nurr1.

Although not all the single substitutions in this region had the desired effect, i.e. still formed a heterodimer, the data show that all the polypeptides retained the ability to promote gene expression as a monomer, and establish the principle that a number of different types of changes can be made to the I-box region and retain this differential activity.

The differences may be any substitution, insertion or deletion which provides for the loss of RXR heterodimerization. Substitutions include substitution of any amino acid (apart from alanine) by alanine, substitutions which result in a change from a positive to a negative charge (e.g. E561K), or vice versa, or changing an uncharged amino acid into a charged or more polar amino acid (e.g. L562K). The alteration of amino acids in a protein by routine protein engineering techniques is well known in the art, and having demonstrated the various changes shown in the accompanying examples, a person of skill in the art will be able to make and test further similar changes without undue burden. Deletions may be of for example from 1 to 10, such as 1, 2, 3, 4 or 5 residues of the I-box region. The deleted residues may be adjacent or located between other I-box residues which are retained. Insertions may be of for example from 1 to 10, such as 1, 2, 3, 4 or 5 residues, of any amino acid, into the I-box region. Where more than 1 residue is inserted, the residues may be contiguous or located between different existing I-box residues. Any one of the 20 naturally occurring amino acids may be inserted. Preferably the inserted residues will be non-aliphatic, e.g. alanine, leucine, valine, isoleucine, glycine, serine, lysine or the like.

Combinations of substitutions, insertions and deletions may be made, e.g. one or more substitutions and one or more deletions. Where a combination of two of these categories of changes is made, the number of amino acid changes may be for example up to a total number of changes from 2 to 10, such as 2, 3, 4 or 5.

Examples of polypeptides of the invention include those in which at least two, preferably at least three adjacent residues are altered, preferably substituted. Such substitutions may be different or the same, e.g. all to alanine.

Specific examples of such polypeptides include the polypeptides Nurr1 KLL(554-556)AAA, GKL(557-559)AAA, PEL(560-562)AAA or P560A.

Substitutions at these positions are demonstrated to abolish the ability of the Nurr1 polypeptide to promote RXR-mediated transactivation. However, the ability of the polypeptides to activate reporter gene expression as monomers is retained.

The invention further provides a vector comprising a nucleic acid sequence encoding a Nurr1 polypeptide of the invention.

The invention further comprises an assay method for determining if an agent is a modulator of Nurr1 activity, said method comprising:

providing a Nurr1 polypeptide of the invention together with a putative modulatory agent; and

determining whether or not said agent is able to modulate the transcriptional activity of said polypeptide.

Agents which modulate transcriptional activity may do this by binding to the polypeptide in a manner which allows the polypeptide to retain DNA binding activity and which results in a loss or increase of transcriptional activation, or the agent may be one that modulates DNA binding activity itself.

Generally, assays of the invention fall under the classes of reporter gene assays, coactivator interaction assays (two-hybrid assays) and DNA binding assays. By modulating the DNA binding activity of Nurr1 polypeptides of the invention, it may be inferred that the transcriptional activity of the protein is affected.

Nurr1 is important in developing dopamine cells and may also have functions in adult dopamine neurons. Thus modulators found by the assay of the invention may have use in the treatment of Parkinson's disease, schizophrenia, drug addiction, attention deficit hyperactivity disorder (ADHD), manic depression, and other conditions related to aberrant activity of dopamine neurons.

### BRIEF DESCRIPTION OF THE DRAWINGS

FIG. 1 shows the structural and functional domains of nuclear receptors including the region important for dimerisation (the I-box). NT, amino-terminal domain; DBD, DNA-binding domain; LBD, ligand-binding domain. The 11 amino acids (SEQ ID NO:1) of the Nurr1 I-box altered in the accompanying examples are shown underlined.

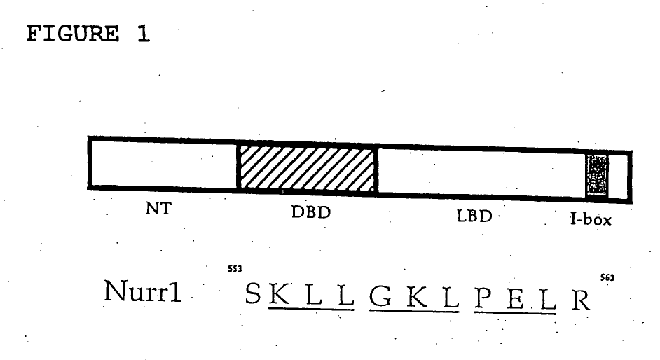

FIG. 2 shows the effect of Nurr1 I-box mutations on the ability to promote RXR-mediated transactivation. The wild-type and mutated Nurr1 derivatives were transfected in JEG3 cells. The ability to transactivate was assessed using a luciferase reporter gene driven by three copies of the hRARβ2 promoter RARE (βRE) upstream of a thymidine kinase promoter.

FIG. 3 shows that the I-box mutations have no effect on the monomeric activity of Nurr1. The monomeric activity was examined in 293 cells. The full-length receptor derivatives were studied using a reporter gene regulated by three copies of the NGFI-B binding site (NBRE; FIG. A).

The activities of the wild-type and mutated ligand-binding domains fused to Gal4 DNA-binding domain were examined on a reporter containing four Gal4-binding sites upstream of a minimal thymidine kinase promoter (FIG. B). Essentially identical results were obtained when the experiments were carried out in JEG3 cells.

FIG. 4 shows the influence of Nurr1 I-box mutations on interaction with RXR in JEG3 cells. The wild-type and mutated Nurr1 ligand-binding domains were fused to the Gal4 DNA-binding domain and the interaction with RXR fused to the VP16 activation domain was examined as described by Perlmann and Jansson 1995.

FIG. 5 shows the effect of I-box mutations on Nurr1-RXR heterodimerization on DNA. The ability of the Nurr1 derivatives to bind DNA as a heterodimer with RXR was assessed in vitro using the gel-shift assay as described by Castro et al. 1999.

Radioactively labelled hRARβ2 promoter RARE (βRE) was used as the probe.

### DETAILED DESCRIPTION OF THE INVENTION

In the present invention, the Nurr1 polypeptide preferably is the murine polypeptide. The amino acid sequence of the murine polypeptide, which is 598 amino acids in size, is available as Genbank accession number S53744. Other Nurr1 polypeptides are known, and may also be used. For example, the human and rat sequences are highly conserved to the murine sequence and have identical numbering of residues in the I-box region. The human Nurr1, also called NOT, is available as Genbank accession NM006186. Rat Nurr1 (RNR) is available as Genbank accession number U72345. All of these have been cloned and are thus available in the art. Due to the very high level of identity of these polypeptides, they may equally be used in the present invention, and reference herein to Nurr1 includes such other species forms of the polypeptide.

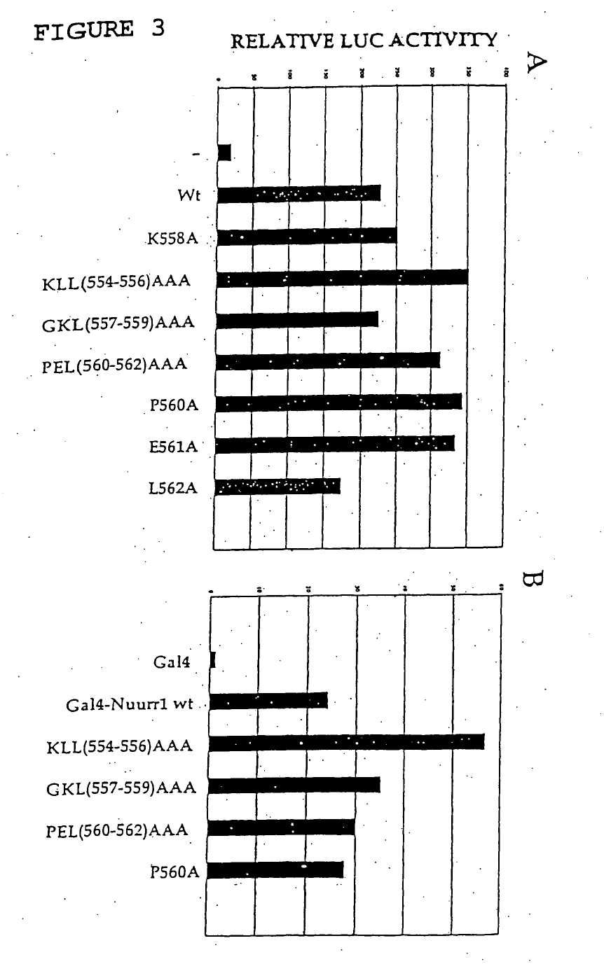

Our data herein indicate that the transcriptional activation activity of the I-box altered Nurr1 is retained by fragments of Nurr1 which have a deletion of their N-terminal region, which also has some transcriptional activating activity. Thus, reference herein to a Nurr1 polypeptide also includes fragments which retain the ability to activate transcription through the ligand binding domain region. Such fragments may comprise at least about 200, preferably at least about 250 amino acids of the full length Nurr1 sequence. This is demonstrated in FIG. 3B herein, where a Nurr1 polypeptide of 246 amino acids is fused to the Gal4 DNA binding domain, and it is shown that this fusion is able to activate transcription from a reporter containing Gal4 binding sites upstream of a tk promoter.

Where Nurr1 polypeptides whose sequences are not on public databases are required, e.g. from other species, these may be obtained by routine cloning methodology. For example, a library of cDNA from a mammalian or other species may be made and probed with all or a portion of a DNA sequence encoding Nurr1 under conditions of medium to high stringency.

For example, hybridizations may be performed, according to standard methods well known as such in the art using a hybridization solution comprising: 5×SSC (wherein SSC is 0.15 M sodium chloride; 0.15 M sodium citrate; pH 7), 5×Denhardt's reagent, 0.5-1.0% SDS, 100 μg/ml denatured, fragmented salmon sperm DNA, 0.05% sodium pyrophosphate and up to 50% formamide. Hybridization is carried out at 37-42° C. for at least six hours. Following hybridization, filters are washed as follows: (1) 5 minutes at room temperature in 2×SSC and 1% SDS; (2) 15 minutes at room temperature in 2×SSC and 0.1% SDS; (3) 30 minutes-1 hour at 37° C. in 1×SSC and 1% SDS; (4) 2 hours at 42-65° C. in 1×SSC and 1% SDS, changing the solution every 30 minutes.

Clones identified as positive may be examined to identify open reading frames encoding Nurr1. It may be necessary to combine more than one clone to achieve a full length open reading frame, as would be understood by the person skilled in art. Clones may then be expressed in a heterologous expression system, e.g. in bacteria or yeast and the protein purified by techniques known in the art.

PCR cloning methods may also be used, based on PCR primers selected for sequences which are conserved between currently known Nurr1 genes found in rat, mouse, human or other species.

In further aspects the invention provides a nucleic acid encoding a Nurr1 polypeptide of the invention, and a vector comprising such nucleic acid. The vector is preferably an expression vector, wherein said nucleic acid is operably linked to a promoter compatible with a host cell. The invention thus also provides a host cell which contains an expression vector of the invention. The host cell may be bacterial (e.g. E. coli), insect, yeast or mammalian (e.g. hamster or human). The vector may be any suitable DNA or RNA vector.

Host cells of the invention may be used in a method of making a Nurr1 polypeptide of the invention as defined above which comprises culturing the host cell under conditions in which said polypeptide or fragment thereof is expressed, and recovering the polypeptide in isolated form. The polypeptide may be expressed as a fusion protein.

Examples of suitable vector systems include bacterial vectors such as pBR322, pUC18 and pUC19, yeast expression vectors, and mammalian vectors, for example vectors based on the Moloney murine leukaemia virus (Ram, Z et al., Cancer Research (1993) 53; 83-88; Dalton and Triesman, Cell (1992) 68; 597-612. These vectors contain the murine leukaemia virus (MLV)enhancer cloned upstream at a β-globin minimal promoter. The β-globin 5′ untranslated region up to the initiation ATG is supplied to direct efficient translation of the cloned protein.

“Operably linked” means joined as part of the same nucleic acid molecule, suitably positioned and oriented for transcription to be initiated from the promoter.

Polypeptides and nucleic acids of the invention may be in isolated form, free or substantially free of material with which they are naturally associated such as other polypeptides or nucleic acids with which they are found in the cell. The polypeptides and nucleic acids may of course be formulated with diluents or adjuvants and still for practical purposes be isolated—for example the polypeptides will normally be mixed with gelatin or other carriers if, used to coat microtitre plates for use in immunoassays. Polypeptides may be glycosylated, either naturally or by systems of heterologous eukaryotic cells, or they may be (for example if produced by is expression in a prokaryotic cell) unglycosylated.

Polypeptides may be phosphorylated and/or acetylated. A polypeptide of the invention may also be in a substantially purified form, in which case it will generally comprise the polypeptide in a preparation in which more than 90%, e.g. 95%, 98% or 99% of the polypeptide in the preparation is a polypeptide of the invention.

Assay methods practiced according to the invention may be arranged in any suitable format known as such to those of skill in the art. For example, an assay may be configured to measure the transcriptional-activating properties of a polypeptide of the invention, by providing a reporter gene construct comprising a promoter region containing one or more (e.g. two, three, four or five) elements to which Nurr1 polypeptides may bind and in doing so, activate transcription of the reporter gene. Examples of suitable constructs are described in the accompanying examples.

For determination of transcriptional activation by a Nurr1-RXR dimer, the hRARβ2 promoter RARE may be used. In contrast, the NGFI-B response element (NBRE) described herein below may be used as a target for monomers of Nurr1 polypeptides to bind to.

These elements may be linked to a suitable transcription initiation region, such at the thymidine kinase gene initiation region, such that transcription is activated by binding of Nurr1 to its cognate binding region(s).

Many suitable reporter genes are known as such in the art, for example luciferase, green fluorescent protein, chloramphenicol acetyl transferase, beta galactosidase, and the like.

Assays of the invention based on the binding of a Nurr1 polypeptide to its cognate DNA binding sequence may be conducted in vitro, and may take any suitable form known to those of skill in the art.

Generally, one or both of the DNA and the polypeptide will carry a detectable label, such as a fluorescent label. The polypeptide and DNA will be brought into contact with each other under conditions suitable for binding to occur, and then the amount of binding in the presence of a putative modulator determined and compared to a suitable control, e.g. the amount of binding in the absence of modulator or a pre-selected modulator with desirable properties.

The binding of DNA to polypeptide can be measured in a number of ways known as such to the skilled person. For example, one or other component may be immobilized on a solid support, and the other brought into contact with it, incubated and then unbound material rinsed away prior to measurement.

One example of an assay format is dissociation enhanced lanthanide fluorescent immunoassay (DELFIA) This is a solid phase based system for measuring the interaction of two macromolecules. Typically one molecule is immobilized to the surface of a multi well plate and the other molecule is added in solution to this. Detection of the bound partner is achieved by using a label consisting of a chelate of a rare earth metal. This label can be directly attached to the interacting molecule or may be introduced to the complex via an antibody to the molecule or to the molecules epitope tag. Alternatively, the molecule may be attached to biotin and a streptavidin-rare earth metal chelate used as the label. The rare earth metal used in the label may be europium, samarium, terbium or dysprosium. After washing to remove unbound label, a detergent containing low pH buffer is added to dissociate the rare earth metal from the chelate. The highly fluorescent metal ions are then quantitated by time resolved fluorimetry. A number of labeled reagents are commercially available for this technique, including streptavidin, antibodies against glutathione-S-transferase and against hexahistidine.

Compounds which target nuclear receptors are of substantial commercial significance in the pharmaceutical industry. Generally, nuclear receptors can be targeted by compounds which are small lipophilic molecules such as steroids, thyroid hormone, retinoids, prostanoids, fatty acids, fatty acid derivatives and numerous small synthetic hydrophobic compounds. Thus potential modulator compounds which may be used in assays of the invention may be natural or synthetic chemical compounds used in drug screening programmes of these classes.

Candidate modulator compounds, including both agonists and antagonists, obtained according to the method of the invention may be prepared as a pharmaceutical preparation. Such preparations will comprise the compound together with suitable carriers, diluents and excipients. Such formulations form a further aspect of the present invention. The formulations may be used in methods of treatment of various conditions associated with aberrant function of dopamine neurons, such as those mentioned herein above.

The amount of putative inhibitor compound which may be added to an assay of the invention will normally be determined by trial and error depending upon the type of compound used. Typically, from about 0.01 to 100 μM concentrations of putative inhibitor compound may be used, for example from 0.1 to 10 nM.

The invention will now be described in detail with reference to the following examples.

### EXAMPLE

### Materials and Methods

The Nurr1 I-box mutants were generated using the GeneEditor™ in vitro Site-Directed Mutagenesis System (Promega) according to the manufacturer's instructions. In short, pCMX-Nurr1 expression vector encoding the full-length mouse Nurr1 was used as the template. Oligonucleotides with 21-40 nucleotides containing the desired mutation were hybridised to the denatured template, extended with T4 DNA polymerase and ligated with T4 DNA ligase. The oligonucleotides used were:

| | | |
|---|---|---|
| for KLL(554-556)AAA: | 5′-CCCAACTACCTGTCTGCAGCGGCGGGGAAGCTGCCAGAAC-3′; | (SEQ ID NO:2) |
| for GKL(557-559)AAA: | 5′-CTGTCTAAACTGTTGGCGGCGGCGCCAGAACTCCGCACC-3′; | (SEQ ID NO: 3) |
| for PEL(560-562)AAA: | 5′-CTGTTGGGGAAGCTGGCAGCAGCCCCGCACCCTTTGCAC-3′; | (SEQ ID NO:4) |
| K558A: | 5′-AAACTGTTGGGGGCGCTGCCAGAACTC-3′; | (SEQ ID NO: 5) |
| P560A: | 5′-TTGGGGAAGCTGGCAGAACTCCGCACC-3′; | (SEQ ID NO: 6) |
| E561A: | 5′-AAGCTGCCAGCACTCCGCACC-3′; and | (SEQ ID NO:7) |
| L562A: | 5′-AAGCTGCCAGAAGCCCGACCCTTTGC-3′. | (SEQ ID NO:8) |

The bases coding for the alanines are underlined. The bacterial colonies obtained after transformation were screened by direct sequencing.

The influence of the I-box mutations on Nurr1 transcriptional activity and on the ability to interact with RXR was examined as described by Perlmann and Jansson 1995, Zetterström et al. 1996, and Castro et al. 1999. Nurr1-RXR heterodimer-mediated transactivation was studied in human chorion carcinoma JEG3 cells. The cells were maintained in minimum essential medium supplemented with 10% fetal calf serum. Transfections were performed in quadruplicates in 24-well plates using the calcium phosphate precipitation method. The cells were plated 24 h prior to the transfection. Each well was transfected with 100 ng of the expression vector for the Nurr1 variants, 100 ng of a reporter plasmid, and 200 ng of βgalactoside plasmid that was used as an internal control for transfection efficiency. The luciferase reporter used was driven by three copies of the retinoid acid response element (RARE) of the human retinoid acid receptorβ2 (hRARβ2) gene promoter (βRE) upstream of the thymidine kinase promoter. 6-8 hours after transfection, the cells received fresh medium supplemented with 10% charcoal-stripped fetal calf,serum and RXR ligand (SR11237;1 μM). The cells were harvested 24 hours late and lysed. The cells extracts were assayed for luciferase and βgalactosidase activity. The ability of the Nurr1 mutants to interact with RXR was examined using the mammalian two-hybrid assay. The ligand-binding domains (amino acids 353-598; LBD) of both wild-type and mutated Nurr1 variants were cloned in frame with the yeast Gal4 DNA-binding domain (amino acids 1-147) in the pCMX-Gal4 expression vector (Perlmann and Jansson 1995). JEG3 cells were cotransfected with pCMX-Gal4-Nurr1-LBD derivatives and pCMX-VP16-RXR containing the herpes simplex VP16 trans-activation domain followed by the complete coding sequence of human RXRα. A reporter gene with four copies of the Gal4-binding sites was used.

The ability of the mutants to activate reporter gene expression as monomers were studied in human embryonic kidney 293 cells. The cells were maintained in Dulbecco's modified Eagles medium with 10% fetal calf serum. Transfections were carried out as with JEG3 cells except that a reporter regulated by three copies of the NGFI-B (nerve growth factor inducible-B)response element (Wilson et al., 1991) (NBREs) was used. The transcriptional activities of the mutated ligand-binding domains were assessed as fusions to the Gal4 DNA-binding domain and a reporter gene with four copies of the Gal4-binding sites was used. All the transfection experiments were performed in quadruplicate dishes and each experiment has been repeated at least twice with essentially identical results. The results of a representative experiment are shown.

The DNA-binding experiments were carried out as described by Castro et al. 1999. Briefly, Nurr1 and RXR proteins for gel mobility shift assays were produced by coupled in vitro transcription and translation in reticulocyte lysates according to the manufacturer's instructions (TNT Quick Coupled Trans cription/Translation System™; Promega). The proteins were incubated in a binding buffer containing 10 mM Tris (pH 8.0), 40 mM KCI, 0.05% NP-40, 6% glycerol, 1 mM DTT, 0.2mg poly(dI-dC) and protease inhibitors. βRE-probe (agcttaaggGGTTCACCGAAAGTTCActcgcat; SEQ ID NO:9) was labelled with ³²P by fill-in reaction using Klenow fragment. After addition of the probe, the reactions were incubated on ice for 20 min. Protein-DNA complexes were resolved by electrophoresis on 4% non-denaturating polyacrylamide gel in 0.5×TBE. After electrophoresis, the gels were dried for autoradiography.

### Results

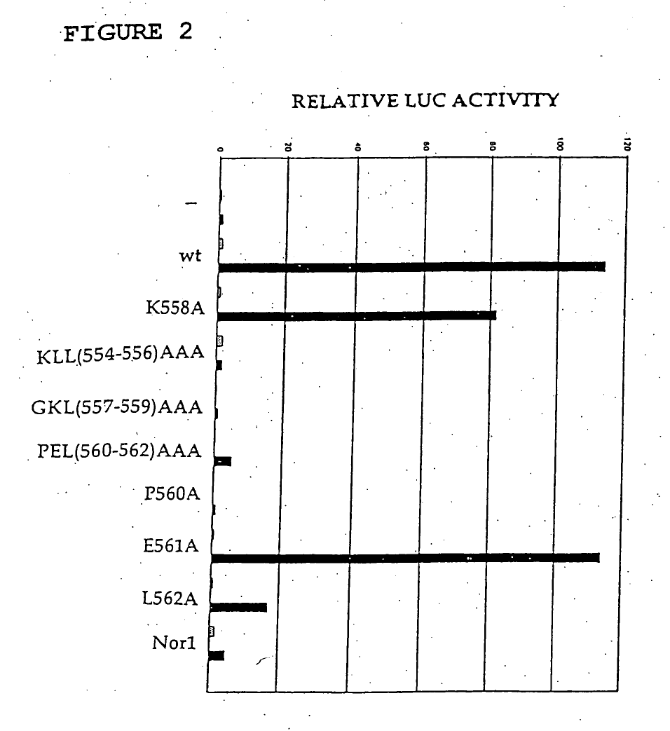

Several of the Nurr1 I-box amino acids were mutated either in combination or individually and the effects of these mutations were studied on RXR dimerization. First, three amino acid alanine substitutions were introduced to the I-box [KLL(554-556)AAA, GKL(557-559)AAA, and PEL(560-562.)AAA]. All these mutations abolished the ability of Nurr1 to promote RXR-mediated transactivation (FIG. 2). The ability to activate reporter gene expression as monomers was, however, intact (FIG. 3). In addition to the triple mutants, we created four individual alanine substitutions (K558A, P560A, E561A, and L562A) in order to examine the contributions of these residues on Nurr1-RXR dimerization. Mutation of the Pro560 to alanine abolished the ability of Nurr1 to activate reporter gene expression as a dimer with RXR but had no effect on monomeric activity (FIGS. 2 and 3A). Conversion of Lys558 to alanine had only a modest effect on Nurr1/RXR heterodimer-mediated transactivation. The ability of Nurr1 to activate the Nurr1-RXR heterodimer-regulated reporter gene was reduced by the substitution of Leu562 by alanine but to a lesser extent than by the P560A mutation. Conversion of Glu561 to alanine had no effect. Thus, the three-residue alanine substitutions and the P560A mutation abolished the ability of Nurr1 to promote RXR-mediated transcription.

N-terminal truncations of the full length protein also retained transcription activation activity (FIG. 3B).

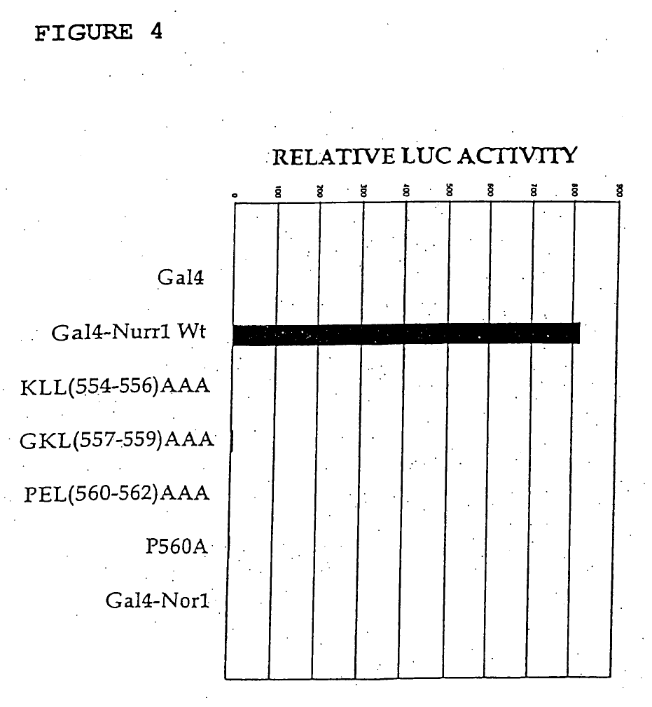

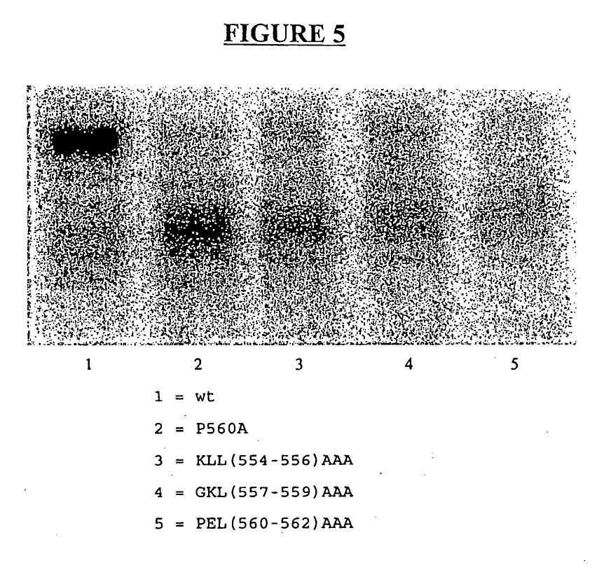

The mutations created could abolish Nurr1-RXR heterodimer-mediated reporter gene expression either by preventing Nurr1 from dimerizing with RXR or by switching Nurr1 into a non-permissive RXR partner. The effects of these mutations on heterodimerization were examined using the mammalian two-hybrid assay. All the mutations that blocked Nurr1-RXR-mediated reporter gene expression also prevented heterodimerization in cells (FIG. 4). The inability of these mutants to heterodimerize on DNA was confirmed in vitro by a gel-shift experiment (FIG. 5). It was also observed that none of the mutations influenced monomeric DNA binding.

In conclusion, four different Nurr1 mutants unable to heterodimerize with RXR were generated. Three of these involved three residue substitutions and one of them was a single amino acid mutation. All these mutants were able to bind DNA and to activate transcription as monomers. Therefore it is unlikely that these substitutions would have had any major effect on the overall structure of the Nurr1 LBD or on the ability to bind a putative ligand. Thus such mutants [e.g Nurr1 KLL(554-556)AAA, GKL(557-559)AAA, PEL(560-562)AAA, and P560A] could serve as useful tools when searching for potential Nurr1 activating ligands. The use of these mutants will allow to differentiate between solely Nurr1 and Nurr1-RXR-heterodimer activating compounds.

### References

Buervenich S, et al 2000 Nurr1 mutations in cases of schizophrenia and manic depressive disorder. Am J Med Genet December 4; 96(6): 808-13.

Castillo S O, et al 1998 Dopamine biosynthesis is selectively abolished in substantia nigra/ventral tegmental area but not in hypothalamic neurons in mice with targeted disruption of the Nurr1 gene. Mol Cell Neurosci 11: 36-46.

Castro D S, et al 1999 Activity of the Nurr1 carboxyl-terminal domain depends on cell type and integrity of the activation function 2. J Biol Chem 274: 37483-37490

Forman B M, et al., 1995 Cell 81: 541-550.

Law S W, et al 1992 Identification of a new brain-specific transcription factor, NURR1. Mol Endocrinol 6: 2129-2135

Lee S -K, et al 1998 Identification of critical residues for heterodimerization within the ligand-binding domain of retinoid X receptor. Mol Endocrinol 12: 325-332

Mangelsdorf D J, et al 1995 The nuclear receptor superfamily: the second decade. Cell 83: 835-839

Millbrandt J 1988 Nerve growth factor induces a gene homologous to the glucocorticoid receptor gene. Neuron 1: 183-188

Ohkura N, et al 1994 Molecular cloning a novel thyroid/steroid receptor superfamily gene from cultures rat neuronal cells. Biochem Biophys Res Commun 205: 1959-1965

Perlmann T and Jansson L 1995 A novel pathway for vitamin A signalling mediated by RXR heterodimerization with NGFI-B and NURR1. Genes Dev 9: 769-782

Perlmann T, et al 1996 Two distinct dimerization interfaces differentially modulate target gene specificity of nuclear receptors. Mol Endocrinol 10: 958-966

Saucedo-Cardenas O, et al 1998 Nurr1 is essential for the induction of the dopaminergic phenotype and the survival of ventral mesencephalic late dopaminergic precursor cells. Proc Natl Acad Sci USA 95: 4013-4018

Truss M and Beato M, 1993 Endocr Rev 14: 459-479.

Wallén \dot{A}. et al 1999 Fate of mesencephalic AHD2-expressing dopamine progenitor cells in Nurr1 mutant mice. Exp Cell Res 253: 737-746

Wilson T E, et al 1991 Identification of the DNA binding site for NGFI-B by genetic selection in yeast. Science 252: 1296-1300

Zetterström R H, et al Retinoid X receptor heterodimerization and developmental expression distinguish the orphan nuclear receptors NGFI-B, Nurr1, and Nor1. 1996 Mol Endocrinol 10: 1656-1666

Zetterström R H, et al 1997 Dopamine neuron agenesis in Nurr-1 deficient mice. Science 276: 248-250

1. A Nurr1 polypeptide which in comparison to a wild-type Nurr1 polypeptide has at least one amino acid difference, which is unable to promote RXR-mediated transactivation but which retains the ability to promote gene expression as a monomer, said difference being in the I-box region.

2. A Nurr1 polypeptide according to claim 1 wherein the difference occurs at one or more I-box residues at residues 524 to 566.

3. A Nurr1 polypeptide according to claim 2 wherein the difference occurs at a residue selected from the positions 554, 555, 556, 557, 558, 559, 560, 561 and 562 of Nurr1.

4. A Nurr1 polypeptide selected from the group Nurr1 KLL(554-556)AAA, GKL(557-559)AAA, PEL(560-562)AAA and P560A.

5. A Nurr1 polypeptide according to any one of the preceding claims wherein the wild-type is murine Nurr1 (Genbank accession number S53744).

6. A nucleic acid sequence encoding a Nurr1 polypeptide of the any one of the preceding claims.

7. An assay method for determining if an agent is a modulator of Nurr1 activity, said method comprising:
providing a Nurr1 polypeptide of any one of claims 1 to 5 together with a putative modulatory agent; and
determining whether or not said agent is able to modulate the transcriptional activity of said polypeptide.

8. An assay according to claim 6 which is a transcriptional activation assay which uses a reporter gene construct.


SEQUENCE LISTING (9 sequences)

SEQ ID NO: 1 - protein, 11 aa, Homo sapiens
SKLLGKLPELR

SEQ ID NO: 2 - DNA, 40 bp, Artificial Sequence
  Oligonucleotide for Kll(554-556)AAA mutation
cccaactacctgtctgcagcggcggggaagctgccagaac

SEQ ID NO: 3 - DNA, 39 bp, Artificial Sequence
  Oligonucleotide for GKL(557-559)AAA mutation
ctgtctaaactgttggcggcggcgccagaactccgcacc

SEQ ID NO: 4 - DNA, 39 bp, Artificial Sequence
  Oligonucleotide for PEL(560-562)AAA mutation
ctgttggggaagctggcagcagccccgcaccctttgcac

SEQ ID NO: 5 - DNA, 27 bp, Artificial Sequence
  Oligonucleotide for K558A mutation
aaactgttgggggcgctgccagaactc

SEQ ID NO: 6 - DNA, 27 bp, Artificial Sequence
  Oligonucleotide for P560A mutation
ttggggaagctggcagaactccgcacc

SEQ ID NO: 7 - DNA, 21 bp, Artificial Sequence
  Oligonucleotide for E561A mutation
aagctgccagcactccgcacc

SEQ ID NO: 8 - DNA, 26 bp, Artificial Sequence
  Oligonucleotide for L562A mutation
aagctgccagaagcccgaccctttgc

SEQ ID NO: 9 - DNA, 33 bp, Artificial Sequence
  Oligonucleotide probe beta-RE
agcttaaggggttcaccgaaagttcactcgcat